(19) United States
(12) Patent Application Publication
(10) Pub. No.: US 2020/0016145 A1
(43) Pub. Date: Jan. 16, 2020

(54) TREATMENT OF CHRONIC TRAUMATIC ENCEPHALOPATHY

(71) Applicant: CTEC LLC, Wheeling, WV (US)

(72) Inventors: Brandon LUCKE-WOLD, Gainesville, FL (US); Ryan TURNER, Morgantown, WV (US); Aric LOGSDON, Seattle, WA (US)

(73) Assignee: CTEC LLC, Wheeling, WV (US)

(21) Appl. No.: 16/507,247

(22) Filed: Jul. 10, 2019

Related U.S. Application Data
(60) Provisional application No. 62/695,989, filed on Jul. 10, 2018.

(51) Int. Cl. A61K 31/47; A61K 9/20; A61P 29/00

(52) U.S. Cl. CPC A61K 31/47; A61P 29/00; A61K 9/20

(57) ABSTRACT

The invention relates to compounds, compositions and methods to effectively treat traumatic brain injury (TBI) and chronic traumatic encephalopathy (CTE). As a result of administering a therapeutically effective amount of 3-phenyl-N-[2,2,2-trichloro-1-[[(8-quinolinylamino) thioxomethyl] amino] ethyl]-2-propenamide and/or guanabenz, the effects of traumatic brain injury are mitigated and/or the development of chronic traumatic encephalopathy is reduced or precluded.

### CROSS-REFERENCE TO RELATED APPLICATIONS

This patent application claims the priority benefit under 35 U.S.C. § 119(e) of U.S. Provisional Application No. 62/695,989 entitled “TREATMENT OF CHRONIC TRAUMATIC ENCEPHALOPATHY” filed on Jul. 10, 2018, the contents of which are herein incorporated by reference.

### 1. FIELD

This invention relates to treatment of chronic traumatic encephalopathy (CTE) and, more particularly, compounds, compositions and methods to stabilize, regress or preclude the development and/or progression of CTE and, in particular, CTE in a patient with traumatic brain injury (TBI).

### 2. DESCRIPTION OF THE PRIOR ART

Traumatic brain injury (TBI) presents a serious health burden, with over 30 million Americans experiencing TBI each year. Even subtle, presumably innocuous mild TBI, which is the most common form of TBI, can develop long-term consequences and contribute to immense healthcare costs. Until recently, the mechanism(s) by which injury expands and progresses over time was poorly understood. Technological advances in research techniques allow for improvements in the investigation into what may contribute to long-term clinical presentation of cognitive decline.

The symptoms of TBI manifest in the disease known as CTE. CTE is a disease that is neurodegenerative and progressive in nature. This disease can present in anyone, especially those susceptible to repetitive head injury, notably soldiers and athletes. Persistent symptoms of CTE include impulsivity, aggression, and motor dysfunction. Long-term symptoms include memory loss and cognitive decline. Post-mortem evaluation of brains from patients with CTE has shown perivascular accumulation of tau neurofibrillary tangles in distinct brain regions, and within the depths of the sulci. Tau neurofibrillary tangles are an aggregated form of the hyper-phosphorylated tau protein regulating microtubule structure.

There has been minimal understanding of the development of CTE as a result of repetitive concussive or sub-concussive injury. As aforementioned, CTE is characterized by the presence of aggregated tau neurofibrillary tangles. It is unclear, however, how TBI leads to the development of the hyper-phosphorylated tau and progression to CTE. Therefore, a dire need exists to determine the mechanism(s) regulating tau phosphorylation and de-phosphorylation (kinases and phosphatases, respectively), in order to elucidate how TBI leads to CTE and, in turn, minimizing or precluding the progression of TBI to CTE.

A mechanism implicated in the regulation of tau phosphorylation has been identified in research conducted for protein aggregation in Alzheimer's disease (AD). One mechanism induced by protein aggregation is the cellular stress response known as endoplasmic reticulum (ER) stress. Markers of ER stress have been shown to be elevated in models of AD and have shown to co-localize, or be in the same cell, with hyper-phosphorylated tau prior to the development of neurofibrillary tangles. Once the tangle is formed, the association with ER stress is lost, likely due to tangle formation representing an irreversible step. From a therapeutic perspective, modulation of ER stress at early time points may present a plausible intervention and protective strategy. In pre-clinical studies of AD, salubrinal has demonstrated promising results with respect to modulation of ER stress. Salubrinal is a drug that acts as a specific inhibitor of eIF2α (eukaryotic translation initiation factor 2 α-subunit) phosphatase enzymes, and is a specific inhibitor or ER stress induced apoptosis.

There is a need in the art to develop a method that is effective to treat or mitigate the effects of TBI, in order to treat or mitigate the progression of CTE. Furthermore, there is a need to develop a mechanism to regulate tau phosphorylation and de-phosphorylation such that hyper-phosphorylated tau may be minimized or precluded in order to prevent the development, or mitigate the effects, of TBI and CTE.

### SUMMARY OF THE INVENTION

In one aspect, the invention relates to a method of mitigating traumatic brain injury or reducing development of chronic traumatic encephalopathy in a human. The method includes administering to the human a therapeutically effective amount of 3-phenyl-N-[2,2,2-trichloro-1-[[(8-quinolinylamino) thioxomethyl] amino] ethyl]-2-propenamide having a structure I as follows:

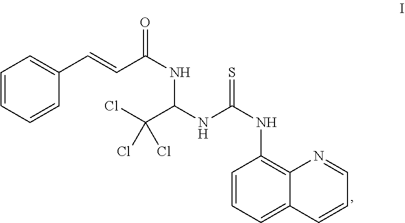 [SMILES: O=C(/C=C/c1ccccc1)NC(NC(=S)Nc1cccc2cccnc12)C(Cl)(Cl)Cl]

or a pharmaceutically acceptable salt thereof.

The step of administering to the human may include one or more techniques selected from the group consisting of injection, parenterally, and orally.

In certain embodiments, the therapeutically effective amount is a daily dosage.

In certain embodiments, a therapeutically effective amount is a dose of 10 mg by intravenous injection, 50 mg by intraperitoneal injection, and 100 mg orally.

In another aspect, the invention relates to a method of mitigating traumatic brain injury or reducing development of chronic traumatic encephalopathy. The method includes preparing a pharmaceutical composition, which includes obtaining an active compound of 3-phenyl-N-[2,2,2-trichloro-1-[[(8-quinolinylamino) thioxomethyl] amino] ethyl]-2-propenamide having a structure I as follows:

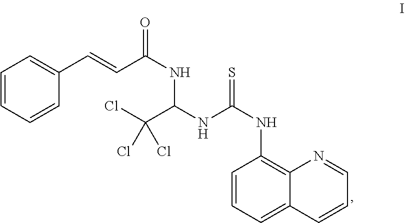 [SMILES: O=C(/C=C/c1ccccc1)NC(NC(=S)Nc1cccc2cccnc12)C(Cl)(Cl)Cl]

or a pharmaceutically acceptable salt thereof;

and combining the active compound with a pharmaceutically acceptable carrier or excipient as mentioned above; and administering a therapeutic effective amount of the pharmaceutical composition to a human having at least one of traumatic brain injury and chronic traumatic encephalopathy.

The pharmaceutically acceptable carrier or excipient may be in a form selected from the group consisting of solid and liquid. The preferred excipient is a solid.

The preferred excipient is 1,4-dihydro-N-methylnicotinic acid (dihydrotrigonelline), which is chosen to enhance blood-brain barrier penetrance.

The carrier or excipient may be selected from the group consisting of inert filler, diluent, binder, lubricant, disintegrating agent, solution retardant, resorption accelerator, absorption agent, coloring agent, and mixtures or combinations thereof. The binder may be selected from the group consisting of starch, gelatin, glucose, β-lactose, corn sweetener, acacia, tragacanth, sodium alginate, carboxymethylcellulose, polyethylene glycol, wax, and mixtures or combinations thereof. The preferred mixture will be lipophilic to enhance penetrance into the brain.

The pharmaceutical composition may be in a form of a tablet.

In certain embodiments, the pharmaceutical composition can include from 0.05% to 95% by weight of the active compound.

The pharmaceutical composition may include an additive selected from the group consisting of medicinal agent, pharmaceutical agent, adjuvant, diluent, vehicle, and mixtures or combinations thereof.

Yet another aspect of the invention is a method of mitigating traumatic brain injury or reducing development of CTE in a human. The method includes administering to the human a therapeutically effective amount of at least one compound selected from the group consisting of:

(i) 3-phenyl-N-[2,2,2-trichloro-1-[[8-quinolinylamino) thioxomethyl] amino] ethyl]-2-propenamide having a structure I as follows:

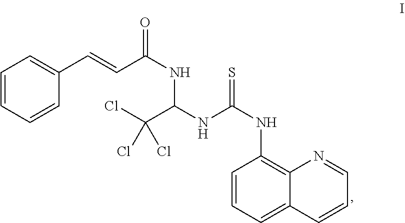 [SMILES: O=C(/C=C/c1ccccc1)NC(NC(=S)Nc1cccc2cccnc12)C(Cl)(Cl)Cl]

or a pharmaceutically acceptable salt thereof, and

(ii) guanabenz having a structure II as follows:

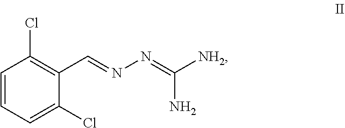 [SMILES: NC(N)=N/N=C/c1c(Cl)cccc1Cl]

or a pharmaceutically acceptable salt thereof.

### BRIEF DESCRIPTION OF THE DRAWINGS

A further understanding of the invention can be gained from the following description of the preferred embodiments when read in conjunction with the accompanying figures.

FIG. 1 are images and plots to show the increased ER stress cascade of the UPR brains of a national football league (NFL) player diagnosed with CTE, WWE wrestler diagnosed with CTE, and control (CTRL) sample wherein Xbox binding protein 1 (XBP1) marks arm 2, p-eIF2α marks arm 1, and ATF6 marks arm 3.

FIG. 2 are images to show ER stress marker inositol requiring enzyme 1 alpha (IRE1α) significantly co-localized with neurofibrillary tangle marker AT270 in the same CTE samples of FIG. 1.

FIG. 3 are images to show increased tau kinase GSK3β in the same areas of pathological tau phosphorylation.

FIG. 4 are images and a plot to show the air acceleration injury model for rats used to generate data.

FIG. 5 are plots that illustrate salubrinal significantly reduced CHOP and GADD34 (markers of ER stress) 24 hours after a single blast injury.

FIG. 6 are images and plots to show protective benefits of post-blast salubrinal administration by immunohistochemistry.

FIG. 7 are images and plots to show salubrinal reduced oxidative stress at 24 hours after injury by altering the ER stress cascade.

FIG. 8 are images that illustrate salubrinal reduced neuroinflammation 24 hours after injury by successfully terminating the ER stress cascade.

FIG. 9 are images and plots to show repetitive blast caused an increase in tauopathy markers AT8 and AT270 at one month, following final injury in the contre-coup brain hemisphere (contralateral).

FIG. 10 are images and plots to show a global inhibitor of ER stress (DHA) inhibited the ER stress activator BiP and tau kinase GSK3β at three weeks following repetitive injury.

FIG. 11 are images and plots to show salubrinal reduced impulsive-like behavior seven days after a single injury.

FIG. 12 are images and plots to show salubrinal reduced impulsive-like behavior by preventing time spent in the open arm of the elevated plus maze 72 hours after the repeat injury.

FIG. 13 are images and plots to show how ER stress inhibition improved cognitive learning after injury measured by Morris Water Maze (panel D) and enhanced retention of a learned event measured by probe trial (panel E).

### DETAILED DESCRIPTION OF PREFERRED EMBODIMENTS

The present invention is directed to compounds, compositions and methods to treat, reduce or preclude the development of chronic traumatic encephalopathy (CTE). It has been found that traumatic brain injury (TBI) may result in the development and progression of CTE. Thus, an object of the invention is to mitigate the progression of TBI, and minimize the development or progression of CTE.

CTE may be characterized by the presence of hyper-phosphorylated tau protein and neurofibrillary tangles. Therefore, the development or progression of CTE may be reduced or precluded by identifying a mechanism to regulate or control the phosphorylation of the tau protein such that hyper-phosphorylation is reduced or precluded. However, in general, protein phosphorylation regulates virtually all biological processes and, although protein kinases are well known drug targets, targeting protein phosphatases has proven to be challenging.

Without intending to be bound by any particular theory, it is believed that endoplasmic reticulum (ER) stress has a role in TBI and the development and progression of CTE. ER is the cellular organelle responsible for protein folding. When the ER becomes stressed due to the accumulation of newly synthesized unfolded or misfolded proteins in the ER lumen, the unfolded protein response (UPR) is activated. The UPR is a signaling mechanism activated in eukaryotic cells in response to ER stress. UPR can restore and maintain homeostasis in the ER to promote cellular survival, or induce apoptosis if ER stress remains unmitigated.

Neuro-inflammation and ER stress are associated with many neurological diseases. Thus, it is an object of the invention to administer to a human, e.g., TBI patient, a therapeutically effective amount of an ER stress inhibitory compound to modulate or inhibit, e.g., reduce or prevent, ER stress and in turn, stabilize, regress or preclude CTE.

In accordance with the invention, a mechanism that leads to development of the neurofibrillary tangles is identified and a pathway that contributes to the tangles is targeted, i.e., ER stress, thereby reducing short-term and long-term symptoms and behaviors in CTE patients. ER stress has three distinct arms or signaling branches that play a role in restoring acute cellular homeostasis after many forms of perturbation. The first, second and third arms are as follows: (1) protein kinase R (PKR)-like endoplasmic reticulum kinase (PERK), (2) inositol-requiring enzyme-1 (IRE-1); and (3) activating transcription factor-6 (ATF6).

In general, ER stress leads to increased binding of the ER chaperone BiP to misfolded proteins in the ER lumen, causing the dissociation of BiP from the ER stress transducers PERK, IRE-1 and ATF6, resulting in their activation. Activated (phosphorylated) PERK phosphorylates eIF2α and thereby attenuates protein translation to relieve the ER workload during stress. In parallel, eIF2α phosphorylation enhances ATF4 translation. ATF4 induces transcription of chaperones and CHOP. CHOP induces expression of GADD34. IRE-1 activation (phosphorylation) causes the splicing of XBP1 mRNA, generating the transcription factor sXBP1.

When ER stress persists or is chronically active, a tau kinase, GSK3β, becomes overactive and serves as a catalyst for tau hyper-phosphorylation, subsequent aggregation and cellular accumulation. This pathway ultimately contributes to neuro-inflammation, which allows the damage to persist and progress over time, and may result in progressive neuro-degeneration.

According to the invention, it has been found that all three arms of the ER stress cascade of the UPR may increase in the brain of a human with CTE, as compared to a human brain without CTE.

In an embodiment of the invention, salubrinal is administered to a human, e.g., patient, to treat or mitigate the effects of TBI, prevent or reduce the likelihood of CTE development, and treat or mitigate the progression of CTE. In general, salubrinal has been primarily experimentally used to study stress responses in eukaryotic cells associated with the action of eIF2. Salubrinal is a selective inhibitor of eIF2 dephosphorylation. There has been research conducted with salubrinal to potentially treat osteoporosis, and accelerate bone healing. “Salubrinal” is used as trade name for the commercially available drug. The chemical name of “salubrinal” used in the present invention is 3-phenyl-N-[2,2,2-trichloro-1-[[(8-quinolinylamino) thioxomethyl] amino] ethyl]-2-propenamide (C₂₁H₁₇C₁₃N₄OS) and the chemical structure is as follows:

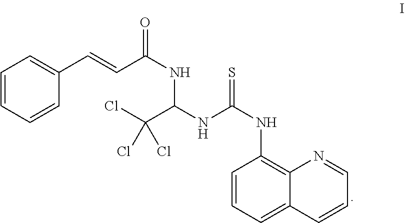 [SMILES: O=C(/C=C/c1ccccc1)NC(NC(=S)Nc1cccc2cccnc12)C(Cl)(Cl)Cl]

In accordance with the present invention, chemical structure I (Compound I) or a pharmaceutically acceptable salt thereof is administered to a patient in a therapeutically effective amount and is used to safely and selectively target the ER stress pathway. The administration of Compound I as a treatment for TBI and CTE can stabilize, regress, reduce or preclude development and progression in the patient.

As will be understood by one skilled in the art, a therapeutically effective amount of Compound I can be administered to a patient by any means known in the art, including but not limited to, injection, parenterally and orally. It is well within the skill of one practicing in the art to determine what dosage, and the frequency of this dosage, which will constitute a therapeutically effective amount for each individual patient.

In certain embodiments, a therapeutically effective amount of Compound 1 is a dose of 1 to 100 mg. In certain embodiments, the therapeutically effective amount of Compound 1 is administered as 10 mg by intravenous (IV) injection, or 50 mg by intraperitoneal (IP) injection, or 100 mg orally. Furthermore, the dosage may be on a daily basis.

The compound(s) of the invention may be formulated in pharmaceutical compositions, which generally include a conventional pharmaceutical carrier or excipient and Compound 1 (or a pharmaceutically acceptable salt thereof) as the/an active agent. In addition, the compositions may include other medicinal agents, pharmaceutical agents, carriers, adjuvants, diluents, vehicles, or combinations thereof. Such pharmaceutically acceptable excipients, carriers, or additives as well as methods of making pharmaceutical compositions for various modes or administration, are well-known to those of skill in the art. The carriers or excipients used are acceptable in the context of being compatible with other ingredients of the composition and must not be deleterious to the patient. The carrier or excipient can be a solid or a liquid, or both, and is preferably formulated with the compound of the invention as a unit-dose composition, for example, a tablet, which can contain from 0.05% to 95% by weight of the active compound. Such carriers or excipients include inert fillers or diluents, binders, lubricants, disintegrating agents, solution retardants, resorption accelerators, absorption agents, and coloring agents. Suitable binders include starch, gelatin, natural sugars such as glucose or -lactose, corn sweeteners, natural and synthetic gums such as acacia, tragacanth or sodium alginate, carboxymethylcellulose, polyethylene glycol, waxes, and the like. Lubricants include, sodium oleate, sodium stearate, magnesium stearate, sodium benzoate, sodium acetate, sodium chloride, and the like. Disintegrators include starch, methyl cellulose, agar, bentonite, xanthan gum, and the like.

Pharmaceutically acceptable carriers and excipients encompass all the foregoing additives and the like.

In certain embodiments the pharmaceutically acceptable excipient s preferably in the form of a solid.

In certain embodiments, the preferred excipient is 1,4-dihydro-N-methylnicotinic acid (dihydrotrigonelline), which is chosen to enhance blood-brain barrier penetrance.

The description provided herein primarily focuses on Compound 1. However, the invention is not limited to the use of Compound 1. It is understood and contemplated that other compounds or compositions that provide the same or similar inhibitory activity (to target the ER stress pathway) as Compound 1 may be used as a substitute or replacement for Compound 1, or a complement with Compound 1. For example, the present invention also includes the use of guanabenz (C₈H₈Cl₂N₄) having the following chemical structure:

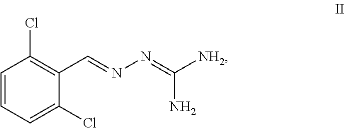 [SMILES: NC(N)=N/N=C/c1c(Cl)cccc1Cl]

or a pharmaceutically acceptable salt thereof.

This compound is available under the trade name WYTENSIN. Compound II is an alpha agonist of the alpha-2 adrenergic receptor, and belongs to the general class of medicines called anti-hypertensives. It is known to use Compound II to treat high blood pressure, e.g., hypertension, by controlling nerve impulses along certain nerve pathways, relaxing blood vessels, and allowing blood to pass through more easily.

While specific embodiments of the invention have been described in detail, it will be appreciated by those skilled in the art that various modifications and alternatives to those details could be developed in light of the overall teachings of the disclosure. Accordingly, the particular embodiments disclosed are meant to be illustrative only and not limiting as to the scope of the invention which is to be given the full breadth of the appended claims and any and all equivalents thereof.

### EXAMPLES

### Role of ER Stress in TBI and CTE

An animal model was used to evaluate TBI, and neuropathological specimens (donated by the Brain Injury Research Institute brain bank) were used to evaluate CTE. TBI was blast induced in the animal model. For the blast-induced TBI (bTBI), test data showed that ER stress was up-regulated acutely, i.e., within the first 24 hours of injury. Salubrinal was administered to the animal model following the injury. The ER stress was modulated by the use of salubrinal. It was found that administration of salubrinal resulted in reducing the markers of neural degeneration and apoptosis.

The administration of salubrinal prior to bTBI was also evaluated in an animal model. Use of the salubrinal ameliorated neuropsychiatric deficits in the form of impulsive-like behavior as measured by the elevated plus maze test/protocol, and spatial memory as measured by the Morris water maze test/protocol.

Using the neuropathological samples for CTE, test data showed that ER stress was significantly up-regulated as compared to brains absent of CTE, i.e., control (“CTRL”) brains, and ER stress was found to be co-localized with hyper-phosphorylated tau.

The results of the bTBI animal models and CTE neuropathological specimens demonstrated that ER stress represents a mechanistic link between TBI and the development of CTE. Further, modulation of ER stress with salubrinal represents a therapeutic approach for mitigating the effects of TBI, and preventing or reducing the likelihood of CTE development and progression.

### Example 1

Human brain specimens were collected from the entorhinal cortex of athletes diagnosed with post-mortem CTE. The specimens were stained for markers of ER stress alone and also co-localized with markers of tauopathy. Traditional immuno-histochemistry was employed with primary antibodies specific for ER stress and pathological tau, and accompanying fluorescent secondary antibodies. Co-localization software (i.e., ImageJ) was used to detect areas of overlap. ANOVA was used for corrected total cell fluorescence analysis. P<0.05 was considered statistically significant. (*=p<0.05, **=p<0.01, and ***=p<0.001).

The results demonstrated that all three arms of the ER stress pathway were increased in human CTE specimens. It was also discovered that ER stress was increased in the same areas where tauopathy was observed, which implicated ER stress in the disease process. This was confirmed by staining for glycogen synthase kinase beta, GSK3β, a catalytic tau kinase associated with ER stress, and finding that it co-localized with tau markers as well.

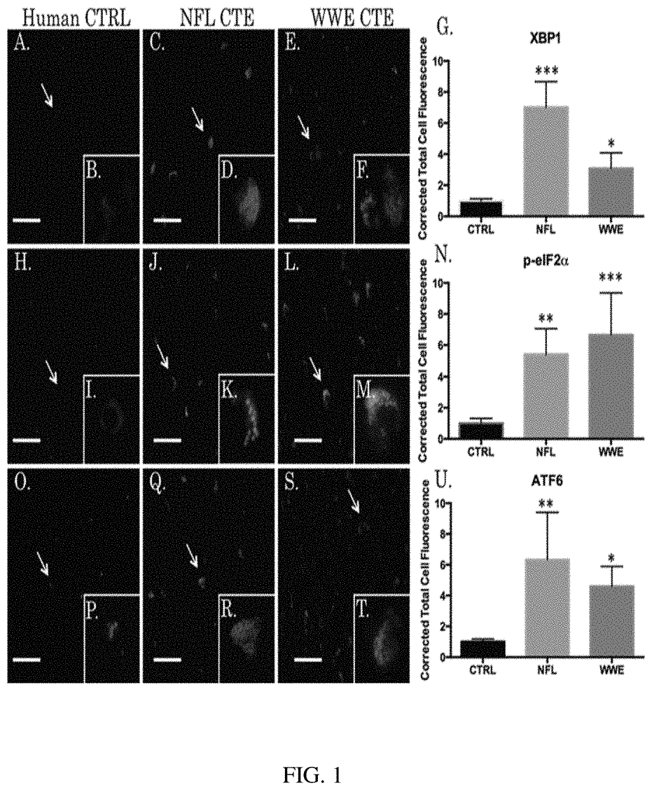

FIG. 1 shows immunohistochemistry results in images A-F, H-M and O-T and corresponding plots G, N and U, respectively, that illustrate all three arms of the ER stress cascade of the unfolded protein response were increased in the brain of a national football league (NFL) player diagnosed with CTE and the brain of a WWE wrestler diagnosed with CTE, as compared to a human control (CTRL) sample absent of CTE. A-G show X-box binding protein 1 (XBP1) increased in CTE samples (2^{nd} arm of ER stress pathway) as compared to human CTRL sample. H-N show phosphorylated elongation initiation factor 2 alpha (p-eIF2α) increased in CTE samples (1^{st} arm of ER stress pathway, and a target of salubrinal) as compared to human CTRL sample. Activated transcription factor 6 (ATF6) also was increased in CTE samples shown in O-U (3^{rd} arm of ER stress pathway) as compared to human CTRL sample. The p-eIF2α is significant because it is a primary target for inhibition.

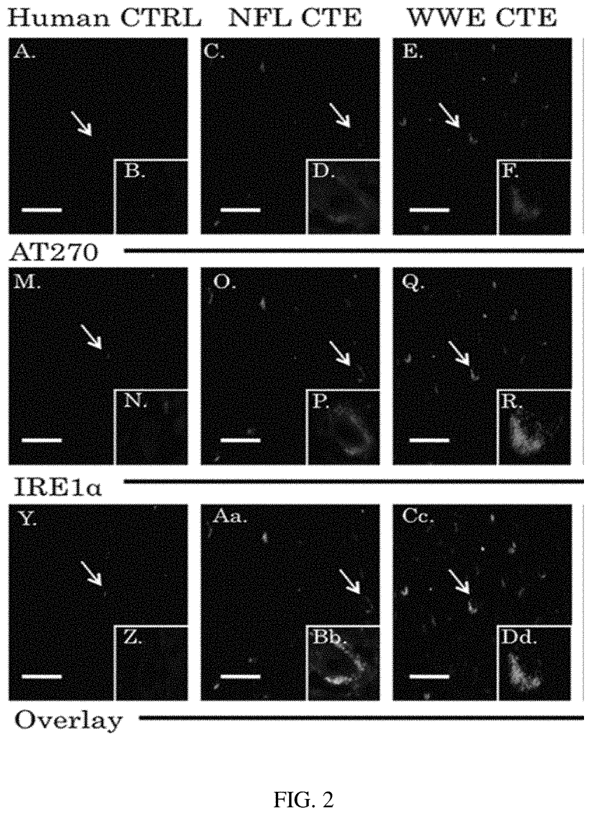

FIG. 2 shows images M-R and A-F that illustrate inositol requiring enzyme 1 alpha (IRE1a) is a marker of endoplasmic reticulum stress that was significantly co-localized with the neurofibrillary tangle marker, AT270, respectively, in the same CTE samples from the NFL player and WWE wrestler and the human CTRL sample. The staining in the overlay panel, images Aa-Dd, suggests that ER stress is associated with tauopathy in the same neuronal cells. The overlap coefficient was ˜0.9 in the brains of both CTE cases for the NFL player and WWE wrestler, indicating a high level of co-localization within the cell.

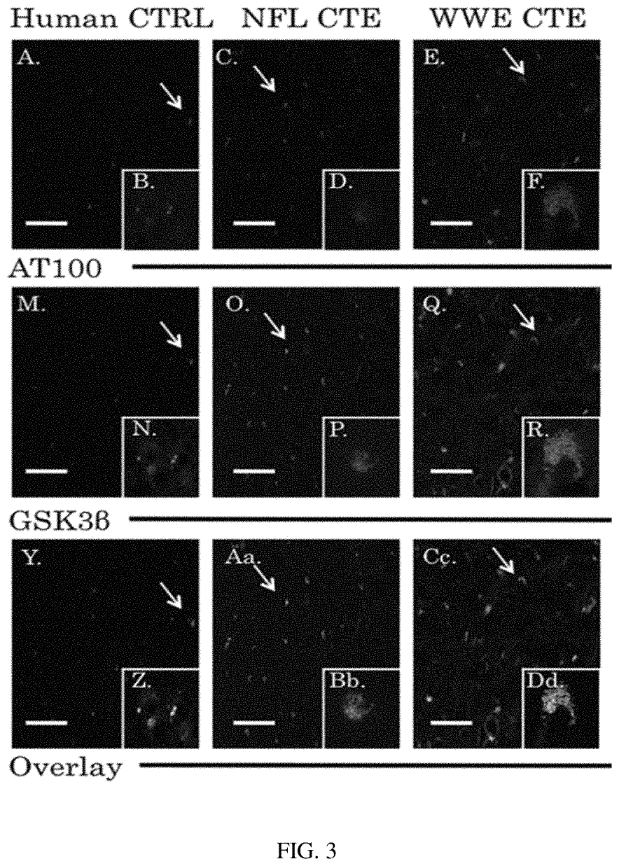

FIG. 3 shows images M-R and A-F that illustrate tau kinase GSK3β was significantly increased in the same areas where pathological tau phosphorylation (AT100) was observed, respectively. The staining in the overlay panel, images Aa-Dd, suggests the association of tau kinase and phosphorylation. The overlap coefficient was 0.88, indicating a high level of co-localization within the cell. Generally, tau kinases causes hyper-phosphorylation of tau, which changes the conformational shape and causes it to accumulate within the cell.

### Example 2

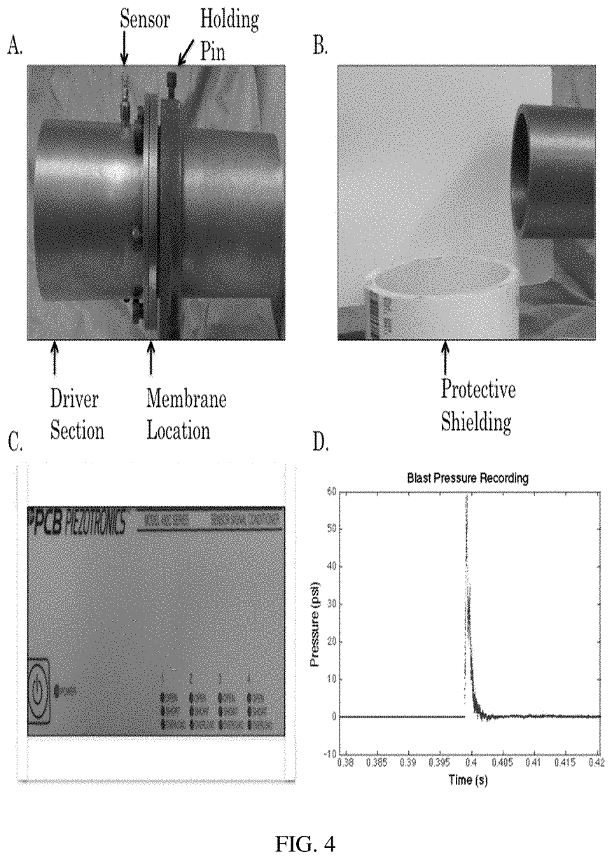

This example assessed the successful targeting of ER stress after TBI. As shown in FIG. 4, images A and B, a tabletop air acceleration injury model was developed. A Sprague Dawley rat was positioned in a protective tube to prevent injury to the peripheral organs, and an acceleration wave was generated and allowed to collide with the skull of the rat. The intensity of the injury was adjusted in a step-wise manner by decreasing or increasing the thickness of the membrane that exploded with pressurized nitrogen gas. An injury paradigm of 50 PSI was selected, which correlates with human concussion, that is the most common type of injury linked to CTE. In FIG. 4, the peak in plot D illustrates the 50 PSI pressure wave. The Sprague Dawley rats were given either one injury, or six injuries over a two-week period. Various time points of sacrifice were chosen to look at markers of ER stress and tauopathy.

Salubrinal was administered to the rats after injury to target ER stress. Salubrinal was administered by IP injection 30 minutes after injury using a dose of 1 mg/kg. It was found that salubrinal inhibited GADD34 to alter ER stress and prevented the surge in pro-death signal CHOP, which reduced GSK3β activity and therefore, prevented an initial tauopathy cascade. Salubrinal-mediated CHOP reduction may be associated with preservation of the pro-form of Caspase-12 that when cleaved, has been associated with ER-mediated apoptosis or cell death. Reducing CHOP with salubrinal also may result in reduction of sustained oxidative stress and neuro-inflammation.

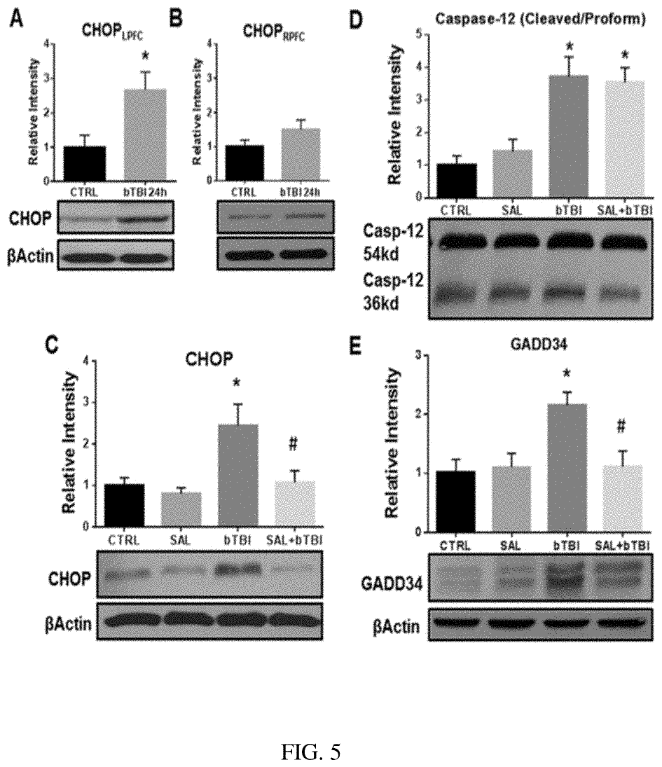

Western blot analysis, immuno-histochemistry, and PCR at various time points after injury were used, as indicated in the FIGS. 5-9. LICOR western blot protocols, IHC world immunohistochemistry protocols, and Applied Biosystems PCR protocols were used for the assays. ANOVA was used for analysis with p<0.05 being statistically significant. *=p<0.05, **=p<0.01, ***=p<0.001. When the drug group was compared to the injury group #=p<0.05, ##=p<0.01, ###=p<0.001.

FIG. 5 shows plots that illustrate salubrinal (SAL+bTBI) significantly reduced CHOP (see plot C) and GADD34 (see plot E) that are markers of ER stress, 24 hours after a single blast injury (bTBI24h), thereby effectively terminating the ER stress response.

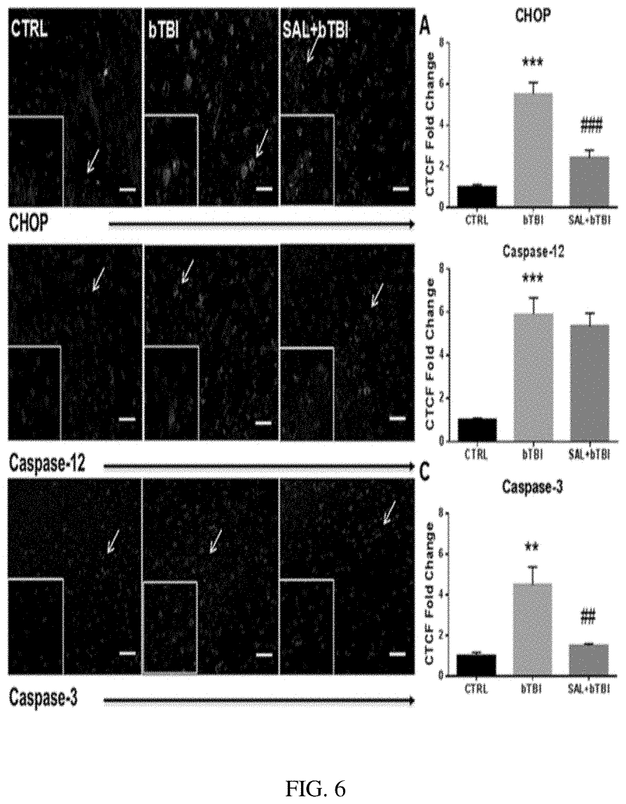

FIG. 6 shows images and plots that confirm the protective benefits of post-blast salubrinal administration (SAL-bTBI) by immunohistochemistry results. CHOP was significantly reduced, which was also associated with a reduction in cleaved Caspase 3 (active form associated with apoptosis). Thus, salubrinal reduced CHOP as well as the pro-apoptotic marker Caspase-3, as shown in plots A and C for blast-induced TBI (bTBI) and administered salubrinal after bTBI (SAL-bTBI).

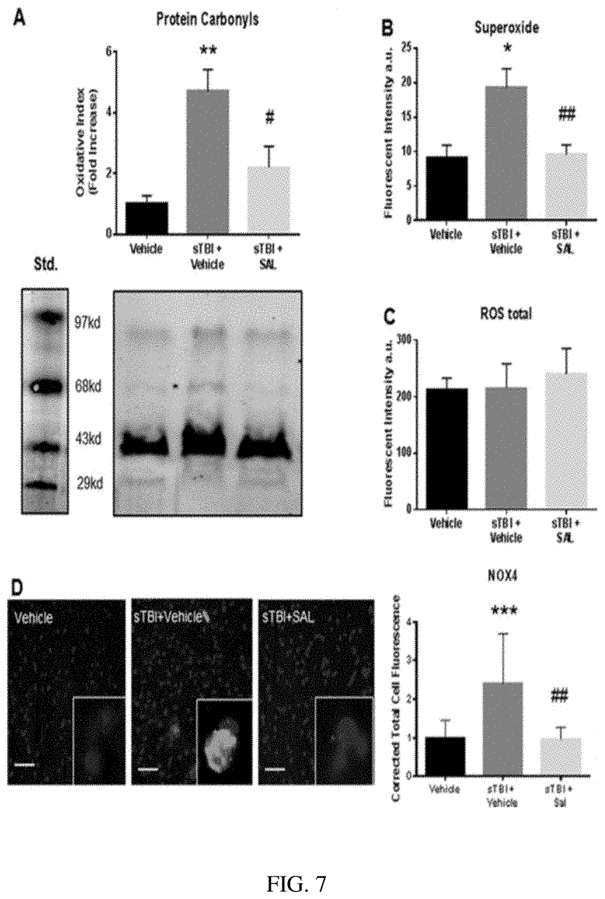

FIG. 7 shows images and plots that illustrate salubrinal reduced oxidative stress at 24 hours after injury by altering the ER stress cascade. Measured components included carbonyls (plot A), superoxide (plot B), reactive oxygen species (ROS) (plot C), and NADPH oxidase 4 (NOX4) (plot D). As shown in A, B and D, administration of salubrinal (sTBI+SAL) reduced protein carbonyls, super-oxides, and total oxidative stress production, respectively.

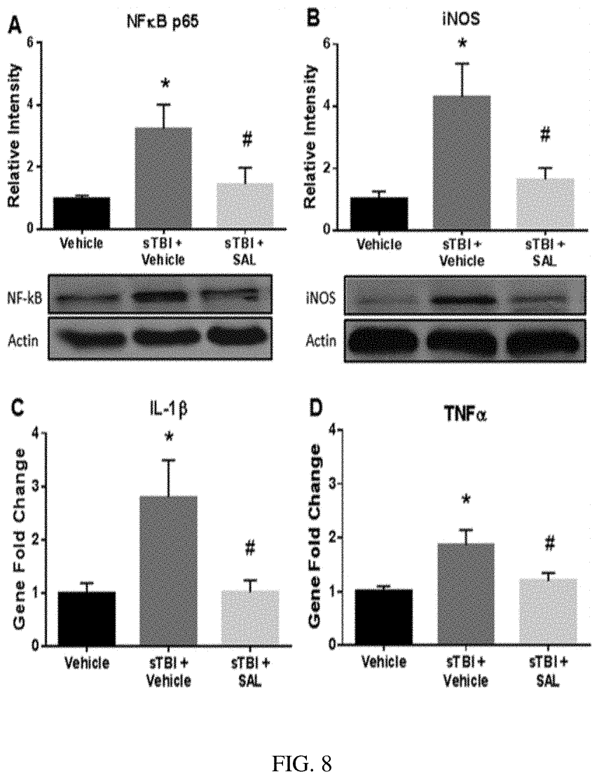

FIG. 8 shows plots that illustrate salubrinal reduced neuroinflammation 24 hours after injury by successfully terminating the ER stress cascade. Measured components included NF kappa B (plot A), inducible nitric oxide synthase (iNOS) (plot B), interleukin 1 beta (IL-1β) (plot C), and tumor necrosis factor alpha (TNFα) (plot D). As shown in plots A, C and D, salubrinal administration (sTBI+SAL) reduced pro-inflammatory markers NFκB, IL-1β and TNFα, respectively.

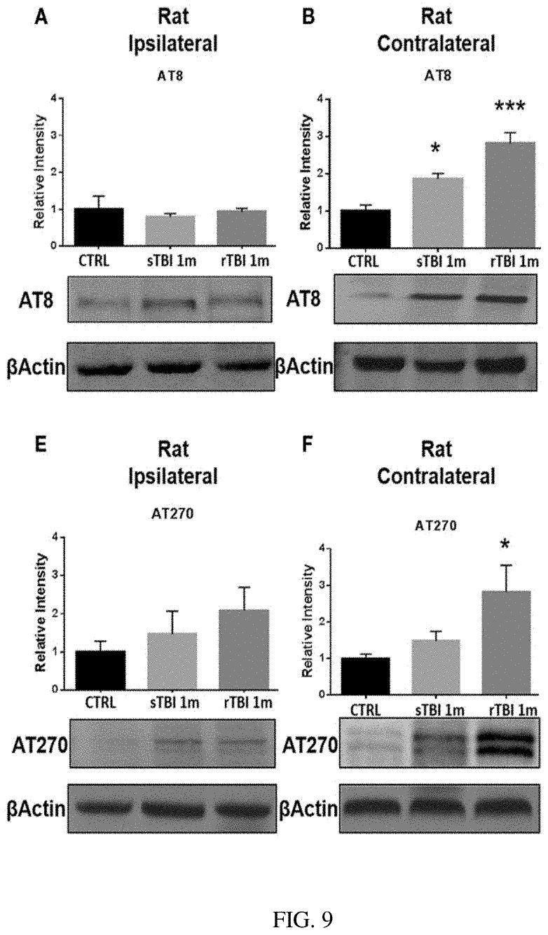

FIG. 9 shows images and plots that illustrate repetitive blast caused an increase in tauopathy markers AT8 (plots A and B) and AT270 (plots E and F) at one month and following final injury in the contre-coup brain hemisphere (contralateral), respectively.

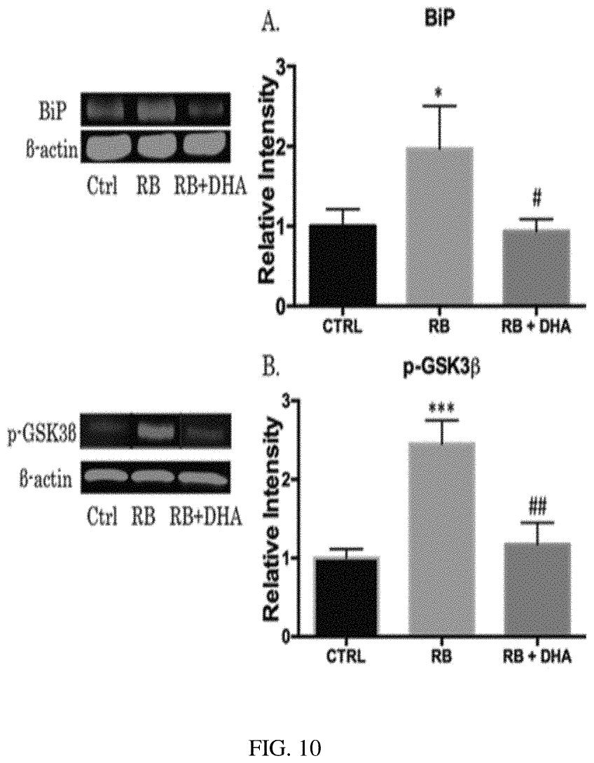

FIG. 10 shows images and plots that illustrate a global inhibitor of ER stress (DHA) inhibited the ER stress activator BiP and tau kinase GSK3β at three weeks following repetitive injury in plots A and B, respectively.

### Example 3

In this example, the effect of targeting (turning off) ER stress on improved behavior was studied by utilizing the injury models and standard protocols for Morris water maze and elevated plus maze. The Morris water maze detected deficits in cognitive performance whereas the elevated plus maze evaluated impulsive-like behavior. The results indicated that targeting ER stress decreased impulsive-like behavior and improved cognitive performance, when provided following the injury. ANOVA was used for statistical analysis with *=p<0.05, **=p<0.01, ***=p<0.001. When drug was compared to injury group #=p<0.05, ##=p<0.01, ###=p<0.001.

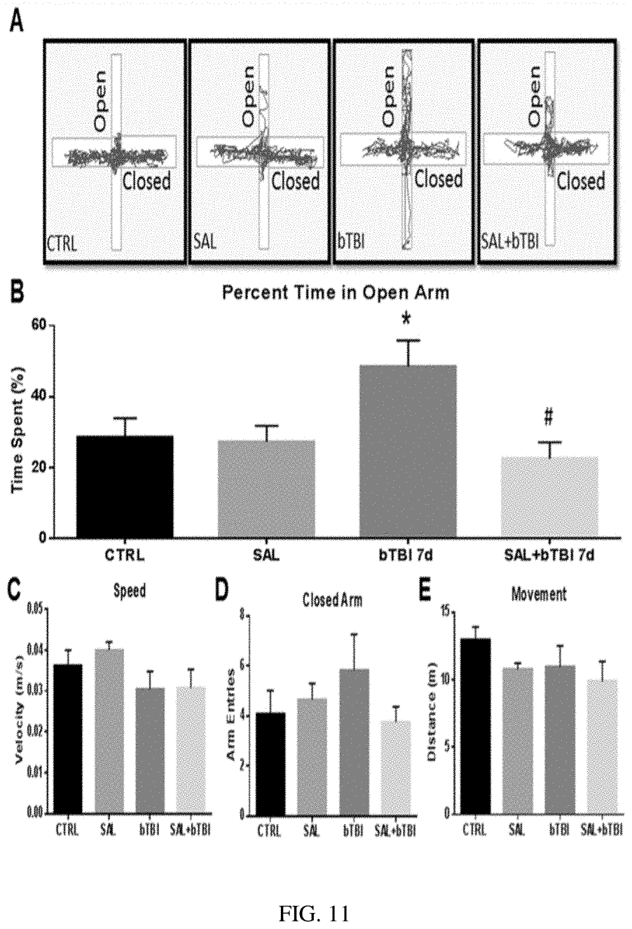

FIG. 11 shows images and plots that illustrate salubrinal administration after injury (SAL+bTBI) reduced impulsive-like behavior seven days after a single injury, as measured by decreased time in the open arm of the elevated plus maze. Plot A shows less time, e.g., travel, in the open arm.

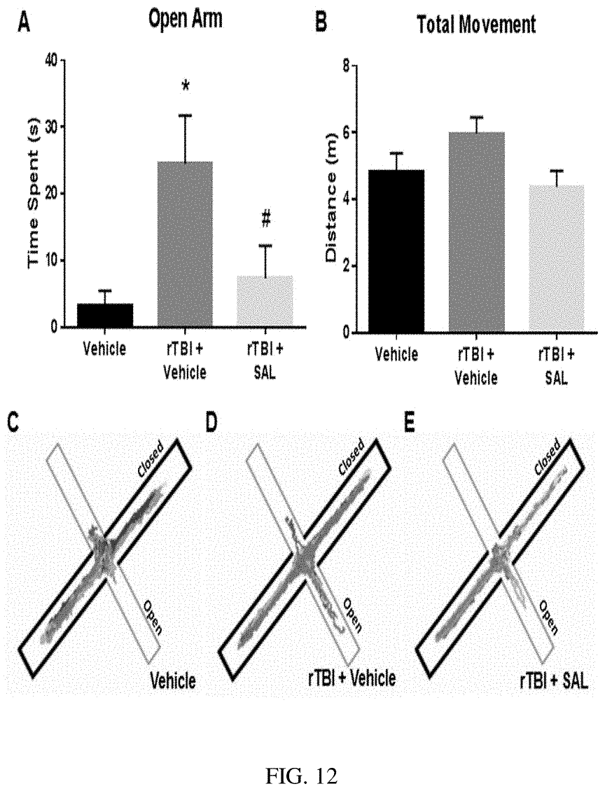

FIG. 12 shows images and plots that illustrate salubrinal administration after injury (rTBI+SAL) reduced impulsive-like behavior by preventing time spent in the open arm of the elevated plus maze 72 hours after the repeat injury.

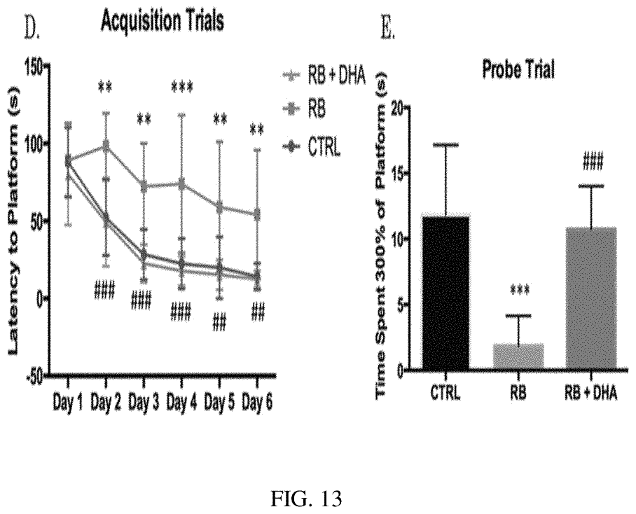

FIG. 13 shows plots that illustrate how ER stress (DHA) inhibition improved cognitive learning after injury measured by Morris water maze (panel D) and enhanced retention of a learned event measured by probe trail (panel E).

### RESULTS/CONCLUSIONS

ER stress was identified as a key pathway in the development of chronic neuro-degeneration following TBI in humans. In the Examples, this pathway was targeted in rodent models (bTBI) and the results demonstrated that subsequent activation of oxidative stress and neuro-inflammation was prevented. By administrating salubrinal to effectively shut-off (terminate) the ER stress pathway, behavior was improved. In particular, inhibiting the ER stress cascade significantly reduced impulsive-like deficits and cognitive decline. The benefit of targeting ER stress by administering salubrinal provides potential diagnostic and therapeutic methods for patients with TBI.

1. A method of mitigating traumatic brain injury or reducing development of chronic traumatic encephalopathy in a human, comprising:
administering to the human a therapeutically effective amount of 3-phenyl-N-[2,2,2-trichloro-1-[[(8-quinolinylamino) thioxomethyl] amino] ethyl]-2-propenamide having a structure I as follows: 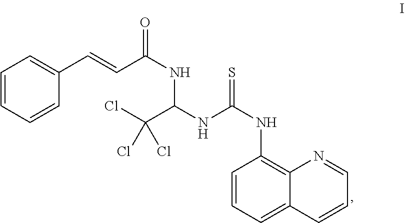 [SMILES: O=C(/C=C/c1ccccc1)NC(NC(=S)Nc1cccc2cccnc12)C(Cl)(Cl)Cl]
or a pharmaceutically acceptable salt thereof.

2. The method of claim 1, wherein the administering comprises one or more techniques selected from the group consisting of injection, parenterally and orally.

3. The method of claim 1, wherein the therapeutically effective amount is a daily dosage.

4. The method of claim 1, a therapeutically effective amount of is a dose of 1 to 100 mg.

5. A method of mitigating traumatic brain injury or reducing development of chronic traumatic encephalopathy, comprising:
preparing a pharmaceutical composition, comprising:
obtaining an active compound of 3-phenyl-N-[2,2,2-trichloro-1-[[(3-quinolinylamino) thioxomethyl] amino] ethyl]-2-propenamide having a structure I as follows: 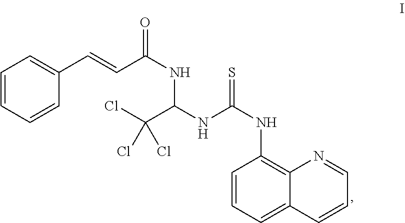 [SMILES: O=C(/C=C/c1ccccc1)NC(NC(=S)Nc1cccc2cccnc12)C(Cl)(Cl)Cl]
or a pharmaceutically acceptable salt thereof; and
combining the active compound with a pharmaceutically acceptable carrier or excipient; and
administering a therapeutically effective amount of the pharmaceutical composition to a human having at least one of traumatic brain injury and chronic traumatic encephalopathy.

6. The method of claim 5, wherein the pharmaceutically acceptable carrier or excipient is in a form selected from the group consisting of solid and liquid.

7. The method of claim 6, wherein the carrier or excipient is selected from the group consisting of inert filler, diluent, binder, lubricant, disintegrating agent, solution retardant, resorption accelerator, absorption agent, coloring agent, and mixtures or combinations thereof.

8. The method of claim 7, wherein the binder is selected from the group consisting of starch, gelatin, glucose, β-lactose, corn sweetener, acacia, tragacanth, sodium alginate, carboxymethylcellulose, polyethylene glycol, wax, and mixtures or combinations thereof.

9. The method of claim 5, wherein the pharmaceutical composition in a form of a tablet.

10. The method of claim 5, wherein the pharmaceutical composition comprises from 0.05% to 95% by weight of the active compound.

11. The method of claim 5, wherein the pharmaceutical composition comprises an additive selected from the group consisting of medicinal agent, pharmaceutical agent, adjuvant, diluent, vehicle, and mixtures or combinations thereof.

12. A method of mitigating traumatic brain injury or reducing development of chronic traumatic encephalopathy in a human, comprising:
administering to the human a therapeutically effective amount of at least one compound selected from the group consisting of:
(i) 3-phenyl-N-[2,2,2-trichloro-1-[[(8-quinolinylamino) thioxomethyl] amino] ethyl]-2-propenamide having a structure I as follows: 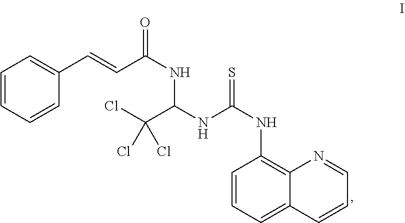 [SMILES: O=C(/C=C/c1ccccc1)NC(NC(=S)Nc1cccc2cccnc12)C(Cl)(Cl)Cl]
or a pharmaceutically acceptable salt thereof, and
(ii) guanabenz having a structure II as follows: 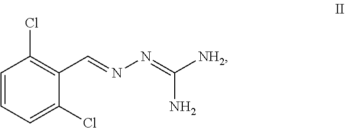 [SMILES: NC(N)=N/N=C/c1c(Cl)cccc1Cl]
or a pharmaceutically acceptable salt thereof.
